(19) United States
(12) Patent Application Publication
(10) Pub. No.: US 2024/0197328 A1
(43) Pub. Date: Jun. 20, 2024

(54) FLOW RESTRICTOR

(71) Applicant: Starlight Cardiovascular, Inc., San Diego, CA (US)

(72) Inventors: Beverly T. TANG, La Jolla, CA (US); Mark JURAVIC, Encinitas, CA (US); Vrad W. LEVERING, Oakland, CA (US); Steven JOHN, San Diego, CA (US); Katie OLSON, San Diego, CA (US); Thomas DUERIG, Fremont, CA (US)

(21) Appl. No.: 18/553,667

(22) PCT Filed: Apr. 1, 2022
(86) PCT No.: PCT/US2022/023060

§ 371 Date: Oct. 2, 2023

Related U.S. Application Data
(60) Provisional application No. 63/170,325, filed on Apr. 2, 2021.

(51) Int. Cl. A61B 17/12; A61B 17/00

(52) U.S. Cl. CPC A61B 17/12036; A61B 17/1204; A61B 17/12109; A61B 17/12172; A61B 17/12177; A61B 2017/00778

(57) ABSTRACT

Devices for restricting flow in blood vessels, and methods of using said devices, the device comprising a first end; a second end; a first opening positioned at the first end, a size of the first opening adjustable from a first size to a second size; a second opening positioned at the second end, the second opening having a greater cross-sectional area than the first opening; and a plurality of struts extending from the first end toward the second end and defining the first opening and the second opening, the plurality of struts extending in a radially outward direction along a portion of their length, wherein the device is collapsible to enable percutaneous delivery.

### CROSS REFERENCE TO RELATED APPLICATIONS

This application claims the benefit of U.S. Provisional Patent Application No. 63/170,325, filed on Apr. 2, 2021, which is herein incorporated by reference in its entirety

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

There are numerous Congenital Heart Defects (CHDs) where blood flow to the pulmonary arteries must be restricted in order to prevent damage to the pulmonary artery bed from sustained pulmonary hypertension. The development of a percutaneous pulmonary flow restrictor could eliminate the need for an open sternotomy surgery on children days to weeks old. The current options involve surgical pulmonary artery banding (PAB) in order to palliate complex ventricular septal defects (VSDs) or atrioventricular septal defects (AVSDs), and hybrid stage I palliation procedure for Hypoplastic Left Heart Syndrome (HLHS). PAB is performed via sternotomy by placing a loop of material around the main or branch pulmonary artery to restrict blood flow. Limitations to the current approach include, but are not limited to, bands being difficult to place precisely to obtain the correct flow profile for the individual patient. Bands are often not adjustable and cannot accommodate a change in blood flow needs over time. Initial placement of the band requires an invasive open surgery. Bands can also interfere with the valve and damage or prevent growth of pulmonary arteries which often necessitates pulmonary artery reconstruction after banding.

### SUMMARY OF THE DISCLOSURE

In a first aspect, a device for restricting flow in a blood vessel is provided. The device comprises a first end; a second end; a first opening positioned at the first end, a size of the first opening adjustable from a first size to a second size; a second opening positioned at the second end, the second opening having a greater cross-sectional area than the first opening; and a plurality of struts extending from the first end toward the second end and defining the first opening and the second opening, the plurality of struts extending in a radially outward direction along a portion of their length, wherein the device is collapsible to enable percutaneous delivery.

In some embodiments, the device is collapsible to enable percutaneous delivery through a 5 F or smaller sheath.

The placement of the device can result in a greater than 30% reduction in pulmonary blood flow. In some embodiments, placement of the device results in Qp:Qs ratios of between 0.8:1 and 2:1. In some embodiments, placement of the device results in arterial oxygen saturations between about 70%-90%.

In some embodiments, the device comprises a length less than about 10 mm.

The device can be configured for placement in branch pulmonary arteries.

In some embodiments, the plurality of struts extend radially outwardly or longitudinally from the first end to the second end.

The plurality of struts can define an opening having a larger diameter than the blood vessel to anchor the device in place.

In some embodiments, the device comprises a membrane covering a portion of an outer surface of the device. The device can comprise a membrane covering an outer surface of the device. The device can comprise a membrane covering an inner surface of the device. The device can comprise a membrane covering an outer surface of the device, wherein the membrane covers a portion of the device configured to contact tissue within the blood vessel. The membrane can comprise ePTFE.

The first end can be the distal end or the proximal end.

The first opening can be expandable. In some embodiments, the first opening is balloon expandable. The first opening can be configured to expand upon absorption of a bioabsorbable component.

In some embodiments, the first opening is tethered to a portion of the frame that expands upon growth of the blood vessel, and wherein expansion of the portion of the frame causes expansion of the tethered first opening.

The device can be generally funnel shaped.

The first opening can be adjustable from an approximate diameter of about 1-3 mm to about 2-5 mm. In some embodiments, the first opening is adjustable from an approximate diameter of about 1-3 mm to a diameter of the second opening. The diameter of the second opening can be about 5-12 mm.

In some embodiments, a profile of the device extends slightly radially outwardly or longitudinally from the second end for a first segment and extends radially inward along a second segment from the second segment. The profile of the device can extend generally longitudinally towards the first end from the second segment.

In some embodiments, the device comprises a flange positioned around the second opening. The device can comprise a plastically deformable component positioned at or around the first opening. In some embodiments, the plastically deformable component comprises a polymer and/or metal alloy. In some embodiments, the plastically deformable component comprises stainless steel.

The device can be percutaneously removable.

In some embodiments, the device is configured to be positioned at a bifurcation between two branch blood vessels.

The plurality of struts can be woven or braided or laser cut.

In some embodiments, the first opening is configured to self-expand as the blood vessel grows larger.

In another aspect, a method for reducing flow in a blood vessel is provided. The method comprises percutaneously advancing a device comprising a plurality of struts disposed between a first end and a second end of the device through the vasculature to a placement site within the blood vessel; and expanding the device from a collapsed configuration to a radially expanded configuration, the device in the radially expanded comprising a first opening at the first end and a second opening at the second end, the second opening comprising a different cross-sectional area than the first opening, wherein the cross-sectional area of the second opening is adjustable.

In some embodiments, the method comprises percutaneously adjusting the cross-sectional area of the second opening. Adjusting the cross-sectional area can comprise increasing or reducing the cross-sectional area.

In some embodiments, percutaneously advancing the device comprises advancing the device through a sheath with a diameter of 5F or less.

The method can comprise percutaneously removing the device.

In some embodiments, the placement site is a pulmonary artery. In some embodiments, the placement site is a branch pulmonary artery.

Placement of the device can result in Qp:Qs ratios of between 0.8:1 and 2:1. Placement of the device can result in arterial oxygen saturations between about 70%-90%.

In some embodiments, adjusting the cross-sectional area comprises straightening struts of the device positioned at or around the second opening. Adjusting the cross-sectional area can comprise using a balloon to expand the second opening. In some embodiments, adjusting the cross-sectional area of the second opening comprises applying energy to the second opening to expand the second opening.

Expanding the device can result in positioning the device at a bifurcation of the blood vessel.

In some embodiments, the method comprises the device self-adjusting as the blood vessel grows and the device expands, increasing the size of the opening.

In yet another aspect, a device for restricting blood flow in a blood vessel is provided. The device comprises a first end; a second end; a first opening positioned at the first end, a size of the first opening adjustable from a first size to a second size; a second opening positioned at the second end, the second opening having a greater cross-sectional area than the first opening; and a plurality of struts extending from the first end toward the second end, the plurality of struts extending in a radially outward direction along a portion of their length, wherein the device is collapsible to enable percutaneous delivery, and wherein the device is configured to be positioned at a bifurcation between two branch pulmonary arteries.

In some embodiments, the first end of the device is configured to be positioned against a junction between the two branch pulmonary arteries. The second end of the device can be configured to be positioned within a main branch upstream of the bifurcation. In some embodiments, the second end of the device is configured to be positioned within a main branch downstream of the bifurcation. The second end of the device can be configured to be positioned within a main branch at the bifurcation.

In some embodiments, the device is collapsible to enable percutaneous delivery through a 5 F or smaller sheath.

Placement of the device can result in a greater than 30% reduction in pulmonary blood flow.

The device can comprise a length less than about 10 mm.

The device can be configured for placement in branch pulmonary arteries.

In some embodiments, the plurality of struts extend radially outwardly or longitudinally from the first end to the second end.

The device can comprise a membrane covering a portion of an outer surface of the device. In some embodiments, the device comprises a membrane covering a portion of an outer surface of the device. The device can comprise a membrane covering an outer surface of the device. In some embodiments, the device comprises a membrane covering an outer surface of the device, wherein the membrane covers a portion of the device configured to contact tissue within the blood vessel.

The first opening can be expandable. In some embodiments, the first opening is balloon expandable.

The device can be percutaneously removable.

In some embodiments, the first opening is configured to self-expand as the blood vessel grows larger.

The plurality of struts can be woven or laser cut.

In another aspect, a device for restricting flow in a blood vessel is provided. The device comprises a proximal end; a distal end; a plurality of struts extending from the proximal end toward the distal end, the plurality of struts configured to anchor against walls of the blood vessel; a membrane structure extending from or near the proximal end of the device, the membrane structure comprising a membrane structure proximal end and a membrane structure distal end, wherein the membrane structure provides a passage for blood flow within the device, and wherein the passage has a greater cross-sectional area at the membrane structure proximal end than at the membrane structure distal end, and wherein the cross-sectional area of the membrane structure distal end is adjustable.

In some embodiments, the device is collapsible to enable percutaneous delivery through a 5 F or smaller sheath.

Placement in the device can result in a greater than 30% reduction in pulmonary blood flow.

The device can comprise a length less than about 10 mm.

The device can be configured for placement in branch pulmonary arteries.

In some embodiments, the plurality of struts extend radially outwardly or longitudinally from the first end to the second end.

The device can comprise a membrane covering a portion of an outer surface of the device. In some embodiments, the device comprises a membrane covering an outer surface of the device. The device can comprise a membrane covering an outer surface of the device, wherein the membrane covers a portion of the device configured to contact tissue within the blood vessel.

In some embodiments, the distal end of the device is configured to be positioned against a junction between the two branch pulmonary arteries. The proximal end of the device can be configured to be anchored within a blood vessel upstream of the bifurcation.

In some embodiments, the membrane structure comprises a support structure positioned at or near a membrane structure distal end. The support structure can be plastically deformable. In some embodiments, the support structure forms a flow restricting portion of the passage into an ovular or duckbill shape.

The membrane structure can comprise a support ring positioned at or near a membrane structure distal end. The support ring can be expandable.

In some embodiments, the membrane structure comprises a valve positioned within the passage. The valve can be a slit, cross-slit, or duckbill valve.

In some embodiments, the membrane structure generally comprises a funnel shape.

The membrane structure can be sufficiently flexible to change orientation due to fluid flow.

The membrane structure distal end can comprise a diameter of about 1-3 mm.

In some embodiments, the device is percutaneously removable.

The membrane structure can fold over an end of the plurality of struts and extends towards another end of the plurality of struts, forming a covering.

In some embodiments, the first opening is configured to self-expand as the blood vessel grows larger.

In a further aspect, a method for reducing flow in a blood vessel is provided. The method comprises percutaneously advancing a device through the vasculature to a placement site within the blood vessel; expanding an anchor portion of the device such that it bears against an inner wall of the blood vessel at the placement site; expanding a membrane structure of the device, the membrane structure connected to the anchor portion of the device at or near a proximal portion of the anchor portion, the membrane structure forming a passageway for blood flow through the device, expanding comprising: expanding a proximal end of the membrane structure to form a proximal opening of the passageway; and expanding a distal end of the membrane structure to form a distal opening of the passageway, the distal opening comprising a smaller cross-sectional area than the proximal opening of the passageway, wherein the cross-sectional area of the distal opening is adjustable.

In some embodiments, the method comprises percutaneously adjusting the cross-sectional area of the distal opening. Adjusting the cross-sectional area can comprise increasing or reducing the cross-sectional area.

In some embodiments, percutaneously advancing the device comprises advancing the device through a sheath with a diameter of 5F or less.

The method can comprise removing the device.

The placement site can be a pulmonary artery. In some embodiments, the placement site is a branch pulmonary artery.

Placement of the device can result in Qp:Qs ratios of between 0.8:1 and 2:1. In some embodiments, placement of the device results in arterial oxygen saturations between about 70%-90%.

Adjusting the cross-sectional area can comprise expanding a strut of the device positioned at or around the second opening. In some embodiments, adjusting the cross-sectional area of the second opening comprises using a balloon to expand the second opening. Adjusting the cross-sectional area can comprise applying energy to expand the second opening.

In some embodiments, the method comprises the second opening self expanding as the blood vessel grows.

In another aspect, a device for reducing blood flow in a blood vessel of a pediatric patient is provided. The device comprises an anchor portion configured to anchor against a wall of the blood vessel; an obstruction connected to the anchor portion and positioned such that it blocks blood flow within the blood vessel, wherein the anchor portion is configured to expand as the blood vessel grows, increasing the flow path around the obstruction.

In some embodiments, the device is collapsible to enable percutaneous delivery through a 5 F or smaller sheath.

Placement of the device can result in a greater than 30% reduction in pulmonary blood flow. In some embodiments, placement of the device results in Qp:Qs ratios of between 0.8:1 and 2:1. Placement of the device can result in arterial oxygen saturations between about 70%-90%

In some embodiments, the device comprises a length less than about 10 mm.

The device can be configure for placement in branch pulmonary arteries.

In some embodiments, the device comprises a membrane covering a portion of an outer surface of the device. The device can comprise a membrane covering an outer surface of the device. In some embodiments, the device comprises a membrane covering an inner surface of the device. The device can comprise a membrane covering an outer surface of the device, wherein the membrane covers a portion of the device configured to contact tissue within the blood vessel. The membrane can comprise ePTFE.

The anchor portion can comprise a plurality of woven or braided or laser cut struts.

In some embodiments, adjusting the cross-sectional area of the second opening comprises applying energy to expand the second opening.

In yet another aspect, a method for reducing flow in a blood vessel is provided. The method comprises percutaneously advancing a device comprising an anchor portion supporting an obstruction through the vasculature to a placement site within the blood vessel; and expanding the anchor portion from a collapsed configuration to a radially expanded configuration to anchor to the blood vessel at the placement site and to define a flow path for blood around the obstruction, wherein the anchor portion is configured to self-expand as the blood vessel grows, increasing a cross-sectional area of the flow path around the obstruction.

The method can comprise percutaneously removing the device.

In some embodiments, the placement site is a pulmonary artery. The placement site can be a branch pulmonary artery.

Placement of the device can result in Qp:Qs ratios of between 0.8:1 and 2:1. Placement of the device can result in arterial oxygen saturations between about 70%-90%.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1D show various views of an embodiment of a flow restrictor.

FIGS. 2A-2D show various views of another embodiment of a flow restrictor.

FIGS. 3A-3D show various views of yet another embodiment of a flow restrictor.

FIGS. 4A-4C show various views of an embodiment of a flow restrictor.

FIGS. 5A-5D show flow patterns in the right pulmonary artery at mid-diastole for native anatomy and various flow restrictors.

FIG. 6 shows another embodiment of a flow restrictor.

FIGS. 7A-7C show various views of an embodiment of a flow restrictor.

FIGS. 7D-F show various views of the flow restrictor of FIGS. 7A-7C in an expanded configuration.

FIG. 8 shows an embodiment of a flow restrictor comprises an obstruction element.

FIG. 9 shows another embodiment of a flow restrictor.

FIGS. 10A and 10B show another embodiment of a flow restrictor positioned at a bifurcation of a blood vessel.

FIGS. 10C-E show an embodiment of a flow restrictor positioned at a bifurcation of a blood vessel.

FIGS. 11A and 11B show embodiments of flow restrictors positioned in branch blood vessel proximal to a bifurcation.

FIGS. 12A-12C show embodiments of valves that can be used in flow restrictors.

FIG. 13 shows an embodiment of a cross-sectional profile of a flow restrictor.

FIG. 14 shows another embodiment of a flow restrictor.

FIGS. 15A and 15B show embodiments of a flow restrictor.

FIG. 16 shows an embodiment of a flow restrictor.

FIG. 17 shows yet another embodiment of a flow restrictor.

### DETAILED DESCRIPTION

Described herein are embodiments of a percutaneous and adjustable branch pulmonary artery blood flow restrictor to replace surgical banding in congenital heart defects (CHD), including Hypoplastic Left Heart Syndrome (HLHS).

Described herein are embodiments of percutaneous and adjustable pulmonary artery blood flow restrictors to replace surgical banding. The devices can address the shortcomings from previous attempts at an internal flow restrictor, by providing: 1) implant delivery via catheter through the femoral vein, 2) the ability to percutaneously increase pulmonary blood flow, 3) reliable flow reduction, 4) a short and well anchored implant, with 5) beneficial hemodynamics, that is 6) surgically removable once the restriction is no longer needed. These pulmonary flow restrictors designed for HLHS can be modified to also replace main pulmonary artery banding, more than doubling the number of patients who could benefit. Embodiments of flow restrictors described herein can produce Qp:Qs ratios between 0.8:1 and 2:1 (or 1:1 and 2:1, etc.), arterial oxygen saturations between 70-90% (or 75%-85%), balanced left and right pulmonary flows, and beneficial hemodynamics.

Embodiments of flow restrictor systems comprise an implant and delivery system. The systems described herein can allow for: 1) delivery through a catheter that fits through a 5F sheath in the femoral vein, 2) accurate deployment without blocking downstream branches, 3) a greater than 30% reduction in distal pulmonary artery pressures, and 4) ability to increase pulmonary artery flow percutaneously.

The embodiments of percutaneous pulmonary artery flow restrictors described herein have the potential to reduce morbidity and mortality in newborns with HLHS by providing a more precise and less invasive palliation procedure. The elimination of cardiopulmonary bypass may potentially reduce length of stay and hospitalization costs.

Embodiments of percutaneous and adjustable pulmonary artery blood flow restrictor described herein can replace surgical banding in pediatric congenital heart defects (CHD), including Hypoplastic Left Heart Syndrome (HLHS). Over 100,000 babies worldwide could benefit from a percutaneous flow restrictor.

Approximately 1,000 Americans are born each year with Hypoplastic Left Heart Syndrome (HLHS), a condition where the left ventricle is severely underdeveloped or barely present. The most common treatment for HLHS is a set of three staged palliation procedures to ensure sufficient blood oxygenation to sustain the patient's life. Babies born with HLHS require the first, stage I, palliation surgery, commonly a Norwood procedure, within days after birth to survive. The Norwood procedure is an impressive advancement in survival for HLHS patients, yet there is room for improvement in survival to stage II palliation as the Norwood procedure subjects a neonate to cardiopulmonary bypass which may have deleterious effects on brain development.

A hybrid (half surgical and half percutaneous) stage I palliation procedure exists as an alternative, which requires stenting the ductus arteriosus and banding the branch pulmonary arteries to prevent pulmonary run-off. Hybrid stage I palliation results are variable amongst institutions, limiting the hybrid procedure's utility as a bypass-free first-line alternative to the Norwood procedure. This variability stems from the difficulty of performing reliable branch pulmonary artery banding, a procedure where bands are sutured around newborn branch pulmonary arteries, only millimeters in diameter, to restrict blood flow. The bands are placed in an invasive, open surgery, and it is difficult to determine whether the bands were tightened properly as the chest is open during surgery, thus making pressures non-physiologic. Finally, bands can inhibit pulmonary artery growth, often necessitating pulmonary artery reconstruction.

Attempts have been made to develop a percutaneous branch pulmonary artery flow restrictor for HLHS by modifying vascular plugs. However, clinical use of these early percutaneous flow restrictors has been limited to case studies due to significant issues with the technology, including the following:
1. Hemodynamic Instability: 6F sheaths used to deliver modified vascular plugs were large and stiff, propping open the valves, causing regurgitation, hemodynamic instability, and sometimes death.
2. Lack of Flow Adjustability: None of the previously-attempted modified vascular plugs allow for increasing the blood flow rate as the baby grows, which is a critical need for neonates with rapidly changing anatomy and physiology.
3. Unreliable Results: Some techniques require cutting a hole in a vascular plug on the table, which could have variable results.
4. Risk of Thrombosis: Modifying vascular plugs, which are designed for vessel occlusion could areas of slow flow that could lead to thrombosis.
5. Blocking Flow to Branches: Vascular plugs are often too long for neonate pulmonary artery branches and may block flow to downstream branches.

A percutaneous flow restrictor, when coupled with a ductus arteriosus stent, would enable a fully percutaneous stage I palliation for HLHS and provide a minimally invasive alternative to the Norwood procedure, eliminating the need for an on-bypass surgery for newborns with HLHS. A flow restrictor designed for HLHS could be used in CHD patients typically requiring main pulmonary artery banding, providing a less invasive procedure for as many as 100,000 babies born each year worldwide.

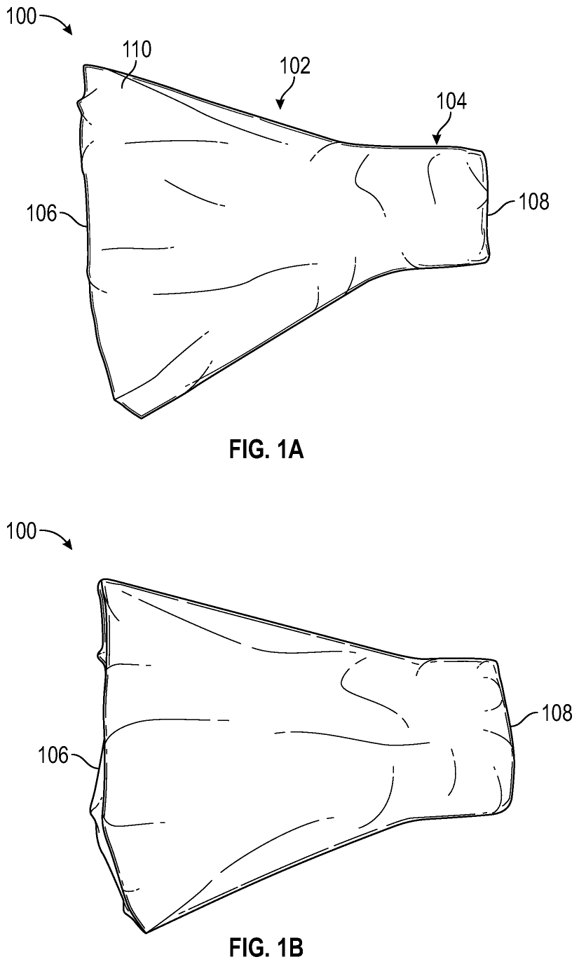

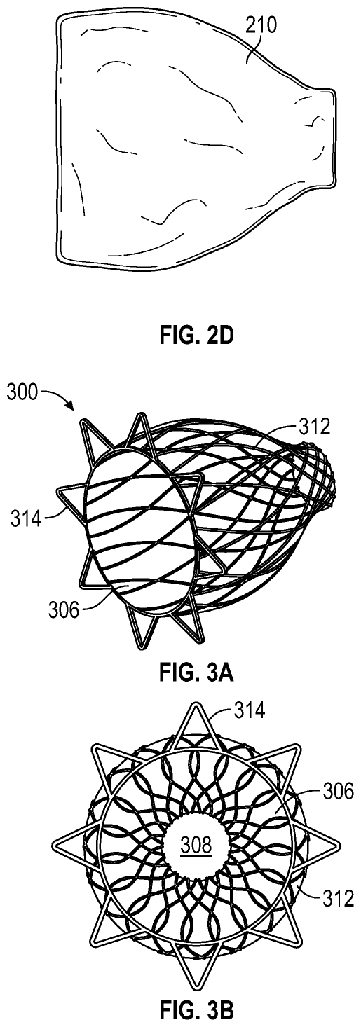

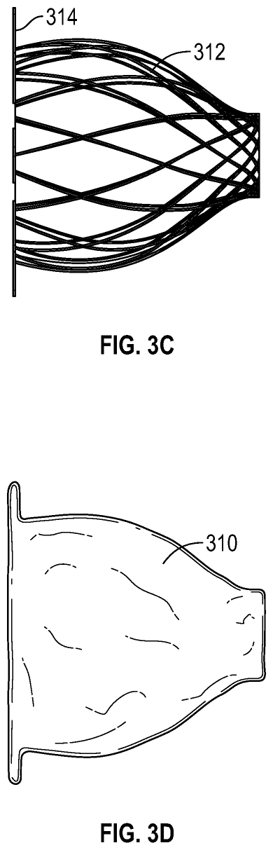

Embodiments herein can comprise any combination of the following features, which have been found to be highly desirable based on extensive input from pediatric interventional cardiologists and cardiothoracic surgeons.
1. Safer catheter-based delivery: The flow restrictor can be delivered through a catheter that is flexible and small enough to cross the valves without causing regurgitation and hemodynamic instability. The delivery catheter can fit through a 5F sheath in the femoral vein to minimize iatrogenic damage to young vessels.
2. Adjustable flow after placement: Pulmonary artery flow can be adjusted (e.g., increased or decreased) percutaneously, for example, with a balloon catheter to dial-in the desired pulmonary artery pressures after initial placement or during a subsequent procedure to accommodate growth (e.g., as shown in FIG. 1C).
3. Reliable results: The flow restrictor can provide adjustability and reliability.
4. Reduced thrombosis risk: The restrictor can produce blood flow patterns that minimize the risk of thrombosis or unwanted vessel remodeling (e.g., as shown in FIGS. 5A-5D).
5. Downstream branch flow ensured by short implant: In some embodiments, the short device length (e.g., <10 mm) can fit within neonate branch pulmonary arteries (e.g., as shown in FIGS. 1A-4B), and placement within the branch pulmonary arteries prevents pulmonary valve interaction. The device shape anchors the implant near the main pulmonary artery to not impede flow in downstream branches, and the implant design may have a self-centering feature to further ensure flow in downstream branches (e.g., as shown in FIGS. 4A and 4B).
6. Removable: Flow restrictor is covered (e.g., by ePTFE) on the abluminal surface of the stent-like structure, which allows the device to be removed in a future surgery without vessel damage

Taken together, these innovations address the shortcomings of earlier attempts at internal flow restrictors.

FIGS. 1A-1C show side views of an embodiment of a flow restrictor 100. The flow restrictor comprises a stent frame that is shape-set. For example, at least a portion of the device 100 can be shape set into a funnel shape. It will be appreciated that a funnel shape can refer to a device shape in which the device comprises a cross-sectional area that generally decreases from one end of the funnel to the other end of the funnel.

The device 100 comprises a first opening 106 at its first end. The first opening 106 comprises a large cross sectional area as compared to the second opening 108 as the second end of the device.

As shown in FIGS. 1A, the device can comprise a funnel shape along a portion 102 of its length. Another portion 104 can have a generally constant diameter or cross-sectional area. In some embodiments, another portion can also have a cross-sectional area that generally decrease from one end to the other end, but at a different rate (e.g., lower rate) than the portion 102.

As described above, the device comprises a larger opening at a first end and, over the length of the device, reduces in diameter, having a smaller opening at a second end. In some embodiments, over the length of the device, the device changes from a diameter of about 5-12 mm to a diameter of about 1-3 mm over a length of about 3-15 mm. Other configurations are also contemplated. For example, over the length of the device, the device can go from a diameter of about 7 mm (or 6-8 mm) to a diameter of about 1.5 mm (or 1-2 mm) over the course of about 3-6 mm. to cover both embodiments. In some embodiments, over the length of the device, the device changes from a diameter of about 10 mm (or about 9-11 mm) to a diameter of about 3 mm (or about 2-4 mm) over the course of 3-10 mm.

In some embodiments, the device 100 comprises a covering 110, as shown in FIGS. 1A-1C. For example, the device 100 can be covered with ePTFE. Other materials are also possible (e.g., nylon, polyurethane, polyester). The covering is on the outer surface of the device, such that it is positioned between the frame and the vessel wall. In some embodiments, there can also be a covering on the inside surface of the device, so that the cover is positioned between the frame and the blood flow.

In some embodiments, the device has a length of about 5-9 mm (e.g., 7.7 mm) long.

The first opening 106 can have a diameter of about 6-9 mm (e.g., 7.5 mm) where it would anchor to the walls of a neonate branch pulmonary artery ˜5-6 mm in diameter via a pressure fit.

Other dimensions are also possible. For example, a length of the device can be about (4-11 mm, about 5-10 mm, about 6-10 mm, about 5-12 etc.). A diameter of the proximal opening of the device can be 5-12 mm.

The second end 108 would restrict blood flow through its about 1-3 mm diameter (e.g., 2.5 mm diameter) (FIG. 1A)

The second opening is adjustable. In some embodiments, the second opening is adjusted during deployment. In other embodiments, the second opening is adjusted after deployment.

The second opening can be enlarged or reduced.

FIG. 1D shows an embodiment of a stent frame 130 positioned within the covering 110. The frame comprises a plurality of struts 132 extending from first end to the second end. The struts 132 are shown as being laser cut, but can be woven or braided, in some embodiments.

In some embodiments, the second opening is balloon expandable. As shown in FIG. 1C, a balloon catheter is inflated to about 3-4 mm (e.g., about 3.5 mm) (FIG. 1C) to plastically deform the smallest ring, and thus the second opening 108 of the flow restrictor to a diameter of about 3-4 mm (e.g., about 3.5 mm) (FIG. 1B), which could be done percutaneously to increase pulmonary artery flow due to the baby's growth. FIG. 1B shows the device with the expanded opening 108. Other adjustment mechanisms are also contemplated, as described in further detail below.

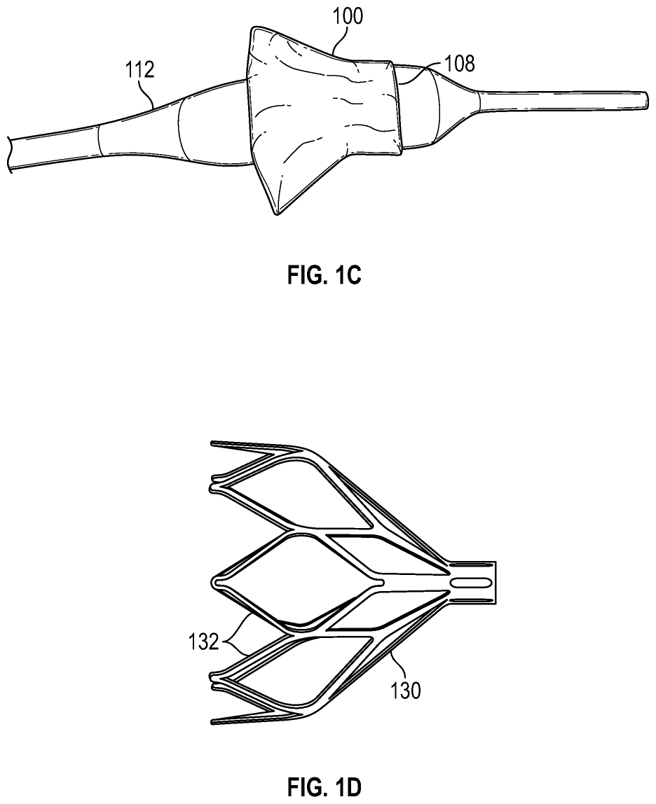

FIGS. 2A-2C show side, perspective, and end views, respectively, of another embodiment of a flow restrictor 200 comprising a funnel-shaped stent frame. The flow restrictor of FIGS. 2A-2C has a more rounded shape than that shown in FIGS. 1A-1C. Furthermore, the Figures show an embodiment where the frame is comprised of a woven structure made of monofilaments (e.g., braided structure) rather than a cut pattern.

The flow restrictor comprises a first opening 206 at a first end of the device and a second opening 208 at a second end of the device. The first opening 206 comprises a larger cross sectional area than the second opening 208.

In some embodiments, from the large diameter end 206, the frame 212 extends slightly radially outwardly or longitudinally for a first segment 202 before extending radially inward along a second segment 204. Finally, the frame 212 extends generally longitudinally towards the small diameter end along a third segment 205.

In some embodiments, an angle formed by the intersection of two struts can be about 50-70°. This orientation between the struts can help to maximize radial force by the struts. In some embodiments, a length of the struts can be selected to create this orientation between intersecting struts.

The flow restrictor can comprise a covering 210 (e.g., ePTFE) on an outer surface of the device, such that it is positioned between the frame 212 and the vessel wall, as shown in FIG. 2D. The flow restrictor can have features and dimensions similar to those described with respect to FIGS. 1A-1C.

The flow restrictor is shown with the covering 210 on the outside of the device in FIG. 2D. FIGS. 2A-2C show the device without the covering. It will be appreciated that the device does not have a covering, in some embodiments. In some embodiments, the covering can be on an outer surface of the device. In some embodiments, the covering can be on an inner surface of the device. In some embodiments the device can have a covering on an inner and outer surface of the device.

The second opening 208 can be adjusted (e.g., balloon expandable) as described with respect the device 100.

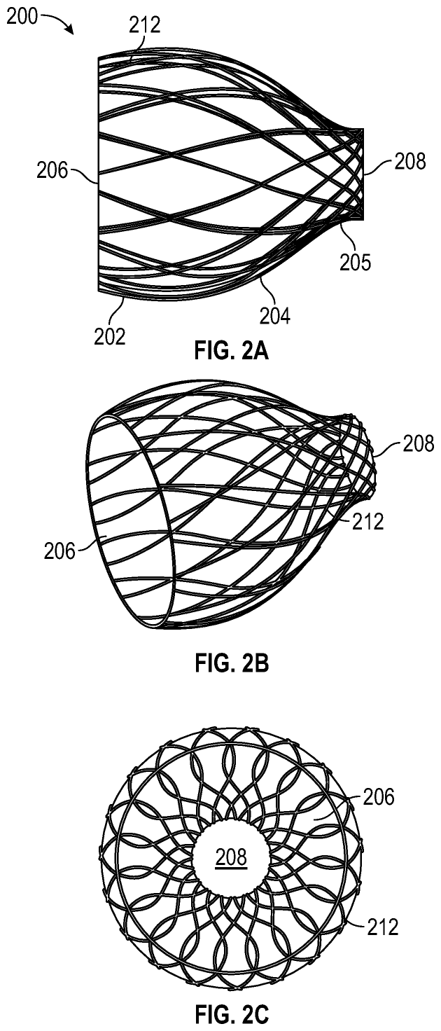

FIGS. 3A-3C show perspective, end, and side views of another embodiment of a flow restrictor 300. The flow restrictor comprises a frame 312 and a covering, similar to device 200.

The device 300 comprises a first opening 306 at a first end of the device and a second opening 308 at a second end of the device end 308, the first opening having a larger cross-sectional area than the first opening. The second opening 308 can be adjustable as described with respect to device 100.

The flow restrictor is shown with the covering 310 in FIG. 3D. FIGS. 3A-3C show the device without the covering on the outside of the device. It will be appreciated that the device does not have a covering, in some embodiments. In some embodiments, the covering can be on an outer surface of the device. In some embodiments, the covering can be on an inner surface of the device. In some embodiments the device can have a covering on an inner and outer surface of the device.

The flow restrictor body also has a similar shape to the flow restrictor 200 shown in FIGS. 2A-2C, but includes a flange 314 around the first opening end 306.

FIGS. 3A-3C show an annular flange 314 comprising multiple petals 316 extending circumferentially along the flange. The petals 316 are shown as comprising two struts meeting at an apex. It will be appreciated that other configurations for the petals or the flange are also possible. For example, the petals con comprise rounded portions. For another example, the flange can comprise arms or segments extending circumferentially along the flange.

The flange 314 can help provide additional anchoring (e.g., in short pulmonary arteries) by extending radially at the ostia of the pulmonary artery bifurcation. In some embodiments, the flange can also be more angled as a flare (e.g., with the flange extending in a proximal direction) such that it provides vessel anchoring via increased tension. The flow restrictor can comprise a covering (e.g., cPTFE). In some embodiments, the flange may also comprise a covering. The covering may help with subsequent removal. The flow restrictor can have features and dimensions similar to those described with respect to FIGS. 1A-1C.

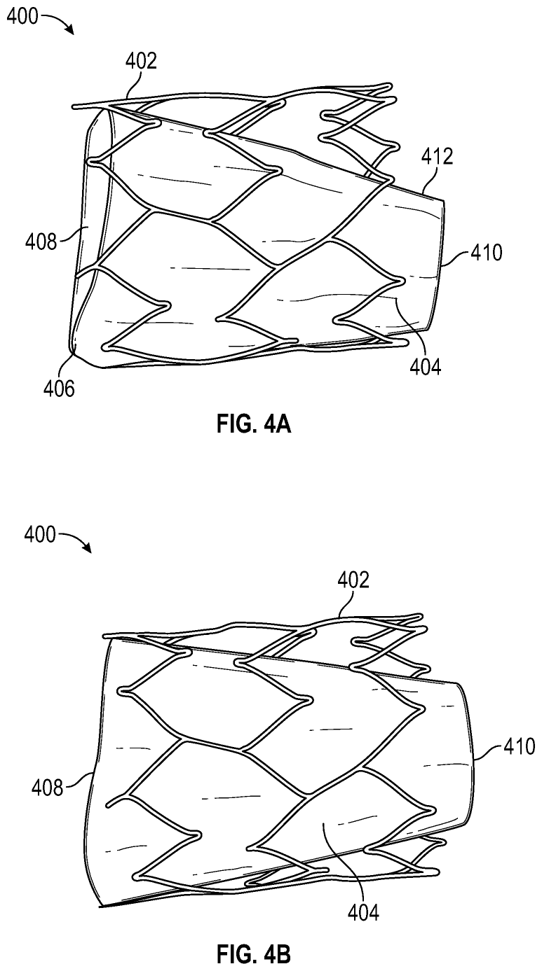

In some embodiments, a balloon can be used to expand the smaller cross-sectional area opening. In some embodiments, balloon expansion breaks or expands stitches restricting the size of the opening. In some embodiments, balloon expansion plastically deforms the opening, for example, straightening one or more struts or segments positioned around and restricting the size of the opening. This concept is described in more detail with reference to FIG. 6.

Other expansion mechanisms are also contemplated. For example, energy (e.g., RF energy) can be used to create holes the covering, or otherwise expand the opening.

As described above, expanding the opening can comprise straightening out one or more struts, segments, rings, or the like positioned around the opening. Referring to FIG. 6, an embodiment of a device 600 comprising struts or segments 602 positioned circumferentially around the device. The struts 602 comprise a plurality of crowns. The crowns can be angular, as shown, or rounded. In some embodiments, the crowns refer to any bends or curves within the strut that can be straightened to increase the circumference or cross-sectional area of the portion of the device defined by that strut. A balloon catheter expanding the smaller opening end would radially expand the strut, causing the crowns to longitudinally contract and radially expand. Expanding the strut can comprise over-expanding them to such that they plastically deform.

In some embodiments, the device comprises struts with varying amounts of crowns, as shown in FIG. 6. A strut with fewer crowns will expand to a smaller diameter than a strut with more crowns. Providing a device with circumferential struts of varying numbers of crowns allows for more control over the shape of the expanded device.

In some embodiments, the device comprises suture rings at different diameters that can be expanded to control the smallest cross-sectional area of the device.

In some embodiments, the frame of the device can be connected (e.g., welded or bonded) to a plastically deformable component (e.g., stainless steel, cobalt chromium, polyethylene, polycarbonate, ePTFE, other metal alloys, other polymers, etc.), which can be balloon expanded to control the flow.

In some embodiments, the flow restrictor 400 comprises a membrane structure 404 with an anchor portion 402, as shown in FIGS. 4A and 4B.

In some embodiments, the membrane structure comprises a thin, biocompatible material (e.g., cPTFE). The membrane structure can extend circumferentially around an inner surface of the blood vessel, forming a passageway for blood to flow. In some embodiments, there are no gaps between the anchor portion 402 and the membrane structure 404. In some embodiments, there may be gaps between the anchor portion 402 and the membrane structure 404.

The membrane structure is formed into a generally tubular shape and tapers as it extends longitudinally, forming a funnel configuration. A proximal end 406 of the membrane structure 404 can be positioned at or near the proximal end of the anchor portion 402. In other embodiment, a proximal end 406 of the membrane structure can be positioned away from the proximal end of the anchor portion.

The proximal end of the membrane structure 404 forms the proximal opening 408 of the passageway formed by the membrane structure, while the distal end 412 of the membrane structure forms the distal opening 410 of the passageway formed by the membrane structure.

The anchor portion 402 can comprise a stent-like cage which can provide a pressure-fit against the walls of the pulmonary artery for device anchoring. The anchor portion 402 can comprise a covering that is positioned between the anchor portion and the vessel wall, as shown in FIG. 4C, which can help ensure removal during a subsequent surgery without damaging the blood vessel. The cage is shown uncovered in FIGS. 4A and 4B so that the funnel-like flow restriction is visible. It will be appreciated that, in some embodiments, the device 400 does not comprise a covering.

In some embodiments, the membrane 404 extends and folds over to cover the stent-like cage 402 at the proximal opening 408 such that the cage is sandwiched between the layers of the membrane.

The membrane structure 404 is attached to the anchor portion 402. The membrane structure 404 can be glued, sutured, heated, sintered, or otherwise bonded to secure it to the anchor portion 402.

The membrane structure 404 can be flexible enough to change orientation due to fluid flow to center itself within the cage and ensure proper flow to downstream pulmonary branches as shown in the shifted position of FIG. 4A relative to 4B. In FIG. 4B, the membrane structure has moved towards the center of the cage lumen.

In some embodiments, the anchor portion has a length of about 5-12 mm (or about 6-11, or about 7-10, or about 7-9, etc.). The membrane structure can have a length of about 1.5-15 mm (or about 2-4 mm etc.). The smallest diameter of the funnel causing flow restriction is about 1-5 mm (e.g., about 2-4 mm, about 2.5 mm, etc.).

In some embodiments, the membrane structure is shorter than the frame. In some embodiments the membrane structure is about the same length as the anchor portion. In some embodiments, the membrane structure is longer than the anchor portion.

The flow restriction portion of the funnel can comprise an expandable structure connected to or inside it. For example, the flow restriction portion can comprise a supporting ring inside, similar to a single ring of a stent. It will be appreciated that the term ‘ring’ does not necessarily refer to a structure having a circular cross-section, but can also comprise structures having ovular or irregular cross-section. This support ring can prevent collapse of the funnel and can be ballooned up in size to increase the diameter of the restriction similarly to the design in FIGS. 1A and 1B, thus increasing pulmonary artery flow as the baby grows. In some embodiments, the support ring can be expanded from a diameter of about 1-3 mm to a diameter of about 4-5 mm. A length of the support ring can be less than or about 1 mm. The covering of the device can be directly attached to this ring, and can either stretch, unfold, or have sutures rupture to expand the diameter of the ring.

The device is delivered through a catheter, which is small and flexible enough to cross two valves without causing simultaneous regurgitation and inducing hemodynamic instability caused by a stiff 6F sheath, while being large enough to enable placement of a covered flow restrictor. The delivery system can comprise an innovative catheter-based delivery system for delivering implants (e.g., nitinol implants) through its lumen, which has thin walls, seamless transition zones, flexibility, and a low kink radius.

In some embodiments, the device is delivered through a sheath without the use of a catheter.

As described above, any of the flow restrictors described herein can comprise a covering (e.g., ePTFE covering, vascular graft covering). The covering can be disposed on at least a portion of an outer surface of the device. In some embodiments, the covering is disposed on at least the portion of the outer surface that is in contact with tissue of the blood vessel wall. Providing a covering between the device frame or anchor portion and the vessel wall helps to prevent tissue ingrowth, which facilitates smoother subsequent device removal.

As described herein, a flow restricting opening of the device can be adjustable (e.g., percutaneously adjustable). In some embodiments, the opening is self-adjusting by using a bioabsorbable restriction (e.g., suture, occlude, etc.). As the bioabsorbable component is dissolved, washed away, or absorbed by the body, the level of flow restriction reduces over time. This can increase pulmonary blood flow, to maintain the desired Qp:Qs ratio even as the baby grows.

In some embodiments, the covering material has an ability to accommodate expansion. The native material of the covering can have stretching properties, enabling expansion. In some embodiments, additional material is cinched into place pre-expansion and then the cinched material being released during expansion. In some embodiments, expansion can cause rupture of sutures holding the covering in a smaller configuration, allowing it to expand. The cover material could also have a porosity to balance the way it anchors into the tissue and limits migration, while still allowing for easy removal from the tissue during a subsequent surgery.

In some embodiments, the flow restrictors can: (1) be deliverable through a 5F sheath or smaller, and (2) ensure approximately 50%±20% (or 50%±10%) reduction in distal pulmonary artery pressures, (3) anchor and fit within the branch pulmonary arteries, (4) not impede flow into the downstream pulmonary artery branches, and (5) enable percutaneous increases in flow and pressure.

### Additional Embodiments of Flow Restrictors

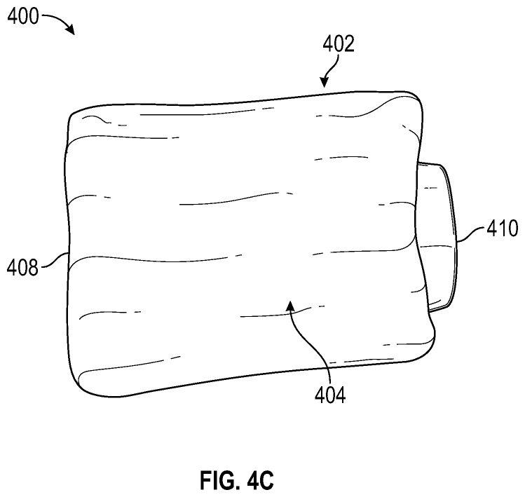

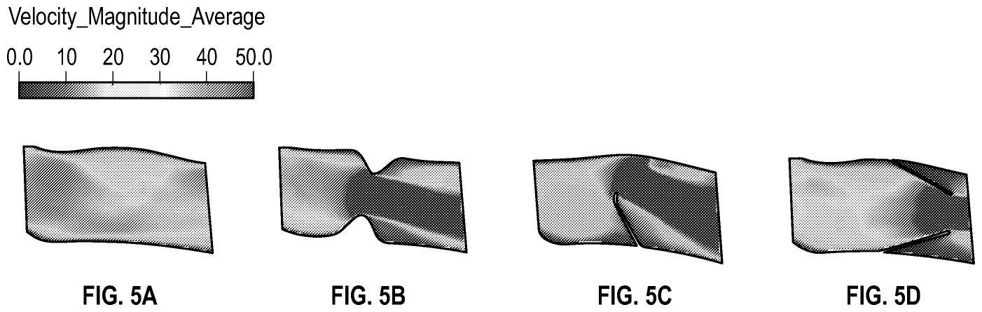

Referring now to FIGS. 7A-7C, front, side, and perspective views of another embodiment of a flow restrictor 700 is shown. The device 700 comprises a constant size obstruction 702 attached to a frame 704 and within the lumen 708 of the flow restrictor. The obstruction 702 remains a constant size as the patient vessels grow. The blood flows around the restriction. As the patient grows, the frame, which is radially expanded against the vessel walls, expands. Because the obstruction 702 does not expand, the area around the obstruction grows, allowing a larger volume of blood flow. Front, side, and perspective views of the expanded device are shown in FIGS. 7D-7F.

The obstruction 702 can comprise a disk, but other shapes (e.g., square, rectangular, triangular, etc.) are also possible. Other configurations for the obstruction are also possible (e.g., a balloon-like structure that is attached to the frame, or a donut-like structure that is attached to the frame and allows flow down the middle of the device lumen.

Struts 706 extend from the obstruction 702 and connect the obstruction 702 to the frame. The struts can be positioned around a circumference or outer perimeter of the obstruction. In some embodiments, the obstruction comprises about 2-14 struts connecting the obstruction to the frame.

In some embodiments, the frame 704 can comprise a stent-like structure. The frame can comprise a covering around its outer surface. The covering can help prevent or minimize contact between the frame and the vessel wall.

In some embodiments the frame can comprise a covering on an interior surface of the frame. In some embodiments, the frame can comprise a covering on a portion of the interior and/or outer surface of the frame.

A size of the obstruction can vary as long as it is sized to produce a Qp:Qs ratio of approximately 0.8:1 to 2:1 (or about 1:1 to 2:1, etc.) as the patient grows, which are clinically acceptable for palliation. In some embodiments, the size can be about 1-10 mm.

In some embodiments, as described herein, the device comprises a constant restriction or occluding size or shape leading to a smaller cross-sectional area opening of the flow restrictor has an adjustable cross-sectional area. This adjustability can be provided by tethering the opening to a portion of the device that is positioned against and radially compressing the vessel wall. As the patient grows and the vessel expands, the portion of the device positioned against the vessel wall will also expand while the restriction size remains the same size, thus automatically increasing the cross-sectional area of the inner opening or flow path.

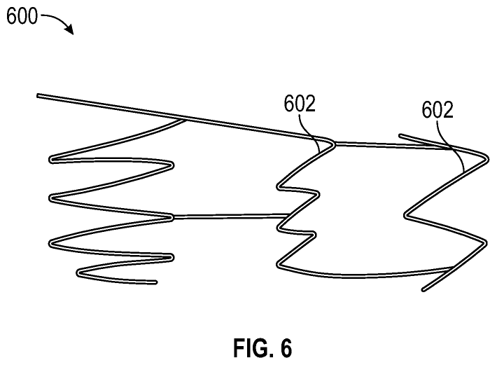

In some embodiments, the obstruction can be centered, as shown in FIGS. 7A-7F. In other embodiments, the obstruction is not centered, and is closer to one side of the vessel wall than another, as shown in FIG. 8. The restriction 802 is positioned closer to one side of the vessel wall, and the flow path 804 is closer to another side of the vessel wall.

In some embodiments, all or a portion of the obstruction is bioabsorbable, causing all or a portion of the obstruction to bioabsorb over time, further increasing the blood flow path.

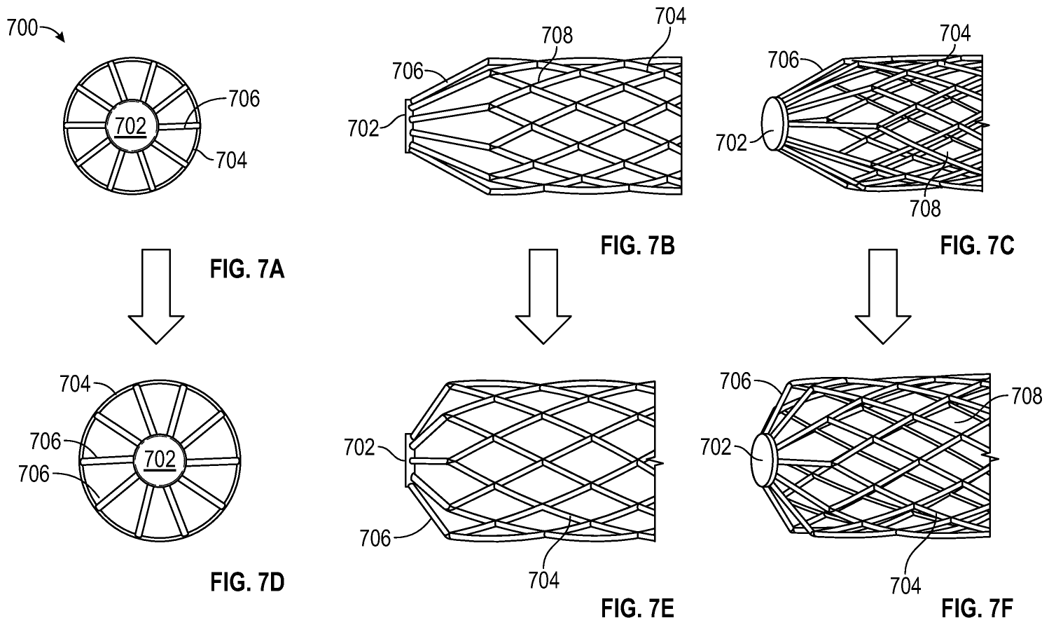

While many of the funnel-shaped device embodiments described herein are described as having the smaller opening at the distal end, it will be appreciated that, in some embodiments, the smaller opening is at the proximal end, as shown in FIG. 9. The flow restrictor 900 has a proximal end 902 with a smaller cross-sectional opening than the distal end 904 opening.

The device 900 can comprise a covering 906 (e.g., ePTFE covering) on an outer and/or inner surface of the device.

In some embodiments, struts 908 at a proximal end of the device can remain exposed to assist in device removal. In some embodiments, the struts 908 extend radially inward at a proximal end of the device to facilitate removal. For example, the struts can form a feature that can be grabbed with a snare. In some embodiments, the device also comprises a loop to facilitate interaction with a snaring device.

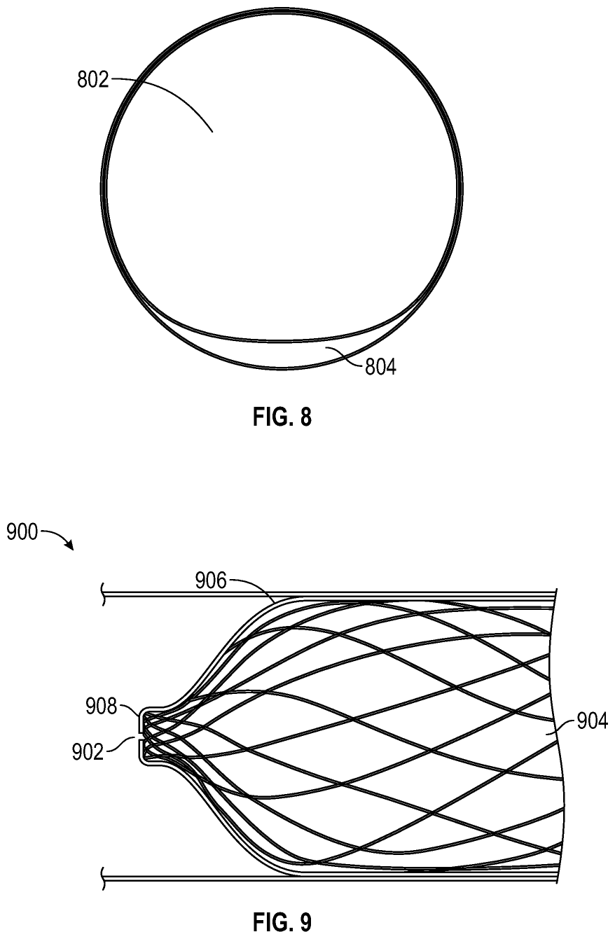

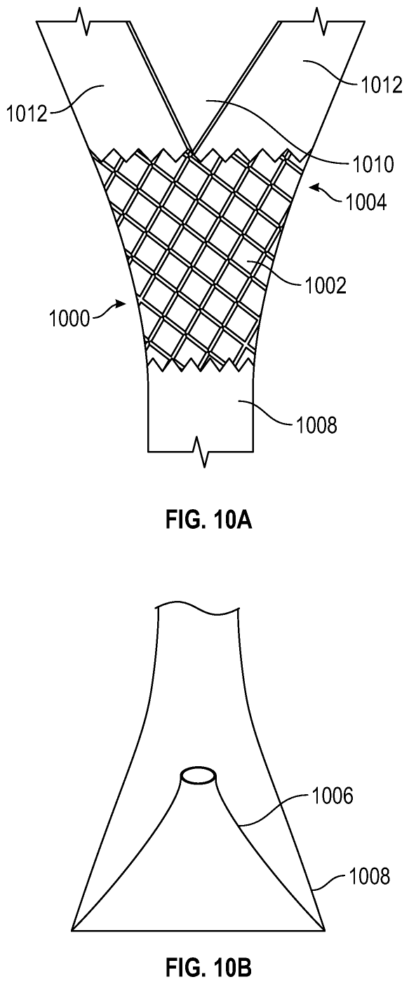

In some embodiments, a pulmonary bifurcation can be used to anchor the flow restrictor. Referring now to FIGS. 10A and 10B, the device 1000 comprises a frame 1002 with a wide distal portion 1004. The width of the frame is sufficient to span both vessels of the bifurcation. The frame can help to anchor the device within the blood vessel. The junction 1010 between the branch vessels 1012 of the bifurcation also anchor the device in place.

The device 1000 can comprise a covering (e.g., ePTFE) covering on some or all of an outer and/or inner surface of the device 1000.

The device comprises an inner membrane structure 1006, as shown in FIG. 10B. The membrane structure can extend circumferentially around an inner surface of the main blood vessel 1008, forming a passageway for blood to flow. The membrane structure is formed into a generally tubular shape with a funnel configuration. A proximal end of the membrane structure can be positioned at or near the proximal end of the frame 1002. In other embodiment, a proximal end of the membrane structure can be positioned away from the proximal end of the frame.

The proximal end of the membrane structure forms the proximal opening of the passageway formed by the membrane structure, while the distal end of the membrane structure forms the distal opening of the passageway formed by the membrane structure.

The distal opening of the passageway can be located proximal to the bifurcation.

It will be appreciated that other devices described herein (e.g., devices, 100, 200, 300, 400) can also be anchored at a pulmonary bifurcation, similar to the device shown in FIGS. 10A and 10B.

FIGS. 10C-E show side and end views of another embodiment of a flow restrictor 1020 positioned at a bifurcation. The flow restrictor 1020 comprises a snare or loop feature 1022 at its proximal end. The flow restrictor 1020 extends radially outwardly from the snare or loop feature 1022 and comprises an umbrella shaped distal end. The distal end is generally rounded and comprises a covering 1024 such as to have minimal interface with the vessel lumen and prevent cellular ingrowth. Struts 1026 extend from the snare or loop feature towards the umbrella shaped distal end.

FIG. 10D shows an end view of the flow restrictor 1020 showing the flow path 1028 surrounding the flow restrictor within the blood vessel 1030. In this embodiment, the flow path will expand as the vessel expands and the covered area remains constant.

FIG. 10E shows an end view of another embodiment of the flow restrictor 1020 comprising holes 1032 in the covering, allowing blood to flow through the covering 1024. In this embodiment, the holes can be enlarged using balloon dilation or other mechanical or electromechanical means.

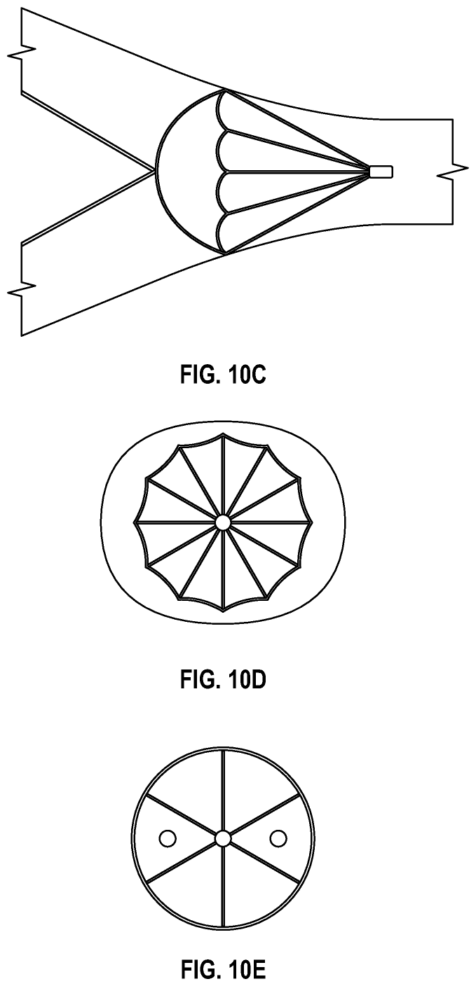

In some embodiments, as shown in FIGS. 11A and 11B, two separate restrictors 1100 can be used in separate branches of a pulmonary bifurcation (e.g., the first two branches of the pulmonary artery branch). In some embodiments, these devices can be tethered together to help them anchor in place. FIG. 11A shows the devices 1100 with the smaller opening positioned at the proximal end. FIG. 11B shows the device 1100 with the smaller opening positioned at the distal end.

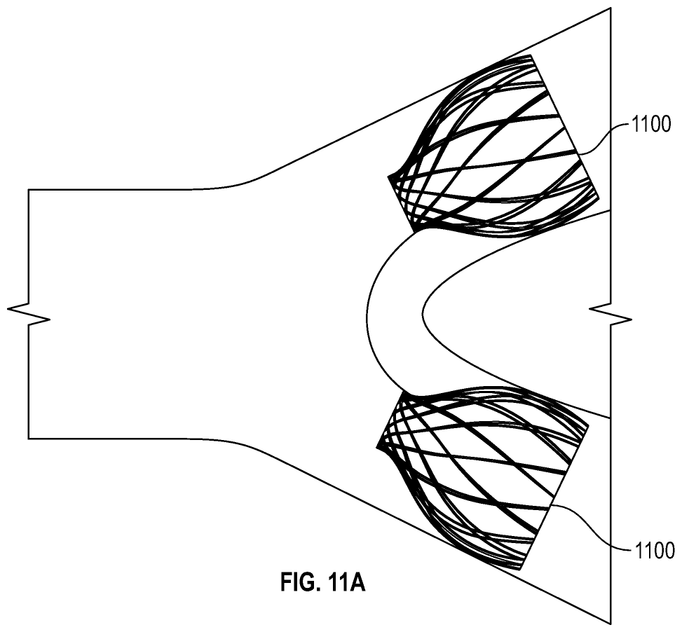

Referring now to FIGS. 12A-12C, in some embodiments, the device comprises a frame (not shown) with a valve positioned within the frame and functioning to restrict blood flow. For example, a duckbill valve 1200 can be used, as shown in the end and side views of FIGS. 12A and 12B, respectively. The opening 1202 of the valve can be balloon expanded to adjust the flow path. Alternatively, sutures along the slit can be expanded or broken to enlarge the cross-sectional area of the opening.

For another example, a cross slit valve 1204 can be used as shown in FIG. 12C. The opening 1206 of the valve can be balloon expanded to adjust the flow path. Alternatively, sutures along the slits can be expanded or broken to enlarge the cross-sectional area of the opening.

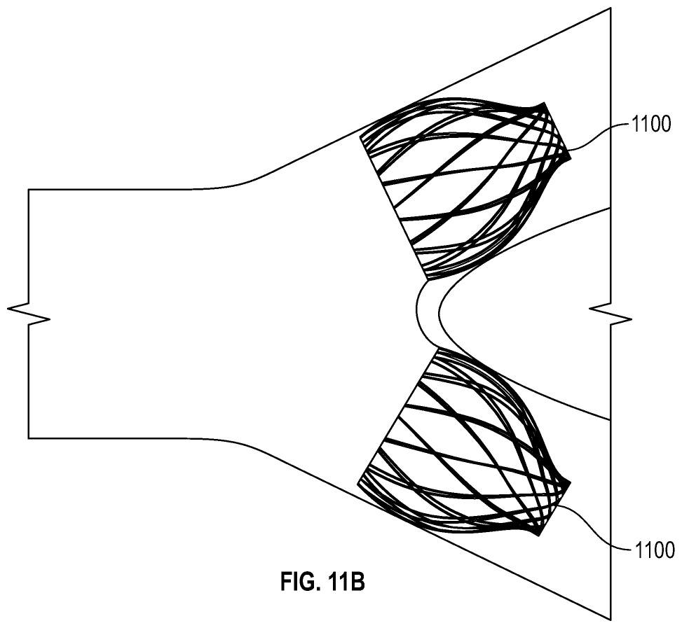

In some embodiments, the flow restrictor comprises an ovular shaped cross-sectional profile, as shown in FIG. 13. This sort of shape distorts the pulmonary artery (e.g., branch pulmonary artery), flattening it, causing the cross-section area to approximate a slit shape, restricting flow.

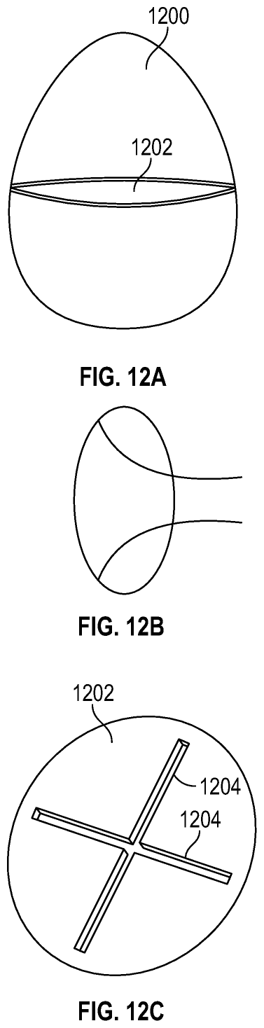

Now referring to FIG. 14, in some embodiments, the flow restrictor 1400 comprises a covered stent comprising several spiraling lateral rings 1402. Twisting the ends of the flow restrictor 1400 in opposite directions can cause a center portion of the device 1400 to constrict, forming a waist that functions to restrict flow. The size of the restriction can be controlled by controlling the amount of twisting the stent during deployment. In some embodiments, the size of the restriction is pre-tensioned prior to deployment.

The waist can later be expanded using balloon expansion, or by deploying a stent within the waist.

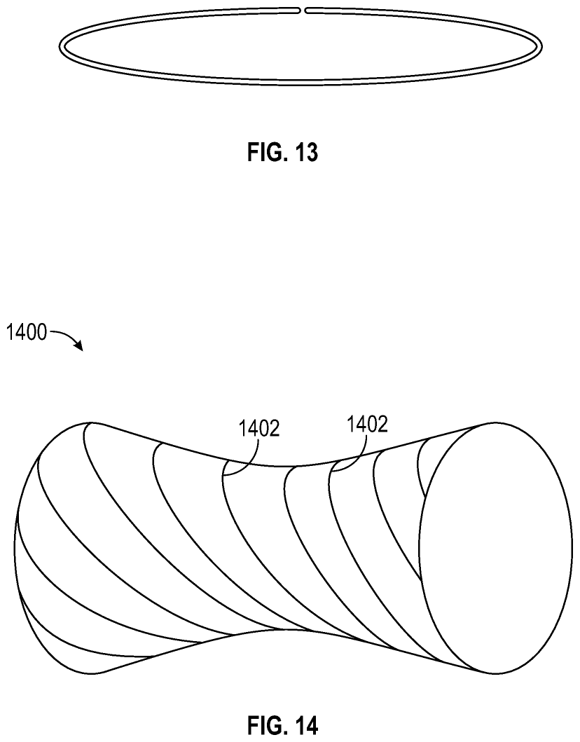

Moving to FIGS. 15A and 15B, in some embodiments, the flow restrictor 1500 comprises two panels 1502, one behind the other. The panels 1502 are connected by a tether or other connecting member 1508. Each panel 1502 comprises a hole or aperture 1504 to allow a flow path. The panels can be twisted to adjust the alignment between the two apertures 1504, thereby adjusting the amount of flow allowed.

In some embodiments, a balloon can be used to adjust the opening between the two panels. The balloon can be used during the initial procedure or during a follow up procedure to adjust the alignment of the panels, thereby adjusting flow.

The panels 1502 can also be connected by a spring 1506, as shown in FIG. 15B. The two apertures can be in opposite positions, as shown, to ensure a non-linear flow path. The distance between the panels 1502 can be adjusted to modulate flow.

In some embodiments, the spring functions as a pressure control valve, to decrease flow as pressure increases. If the proximal panel is fixed in place, the pressure can push the distal panel closer to the proximal panel, making the flow path more tortuous, thereby decreasing flow.

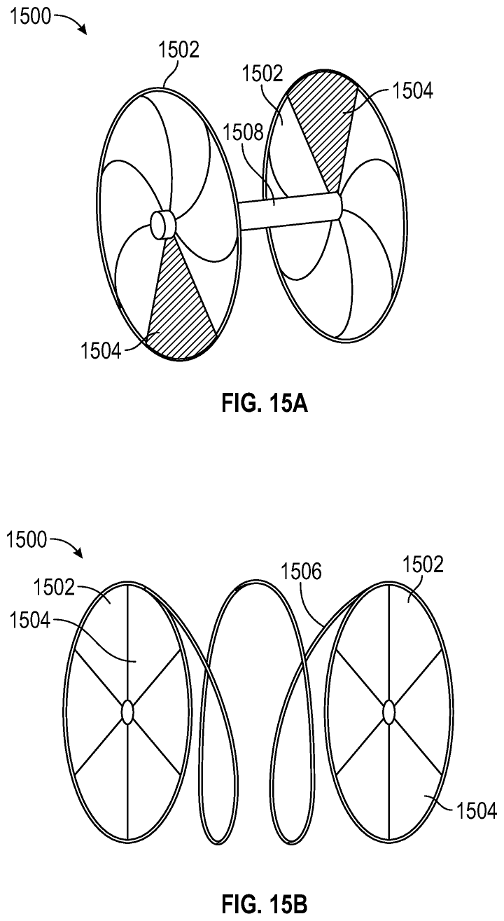

In some embodiments, the flow restrictor comprises a covered frame 1602 defining a helical flow path 1604, as shown in FIG. 16. The length of the helix can be adjusted during deployment or after to adjust the flow.

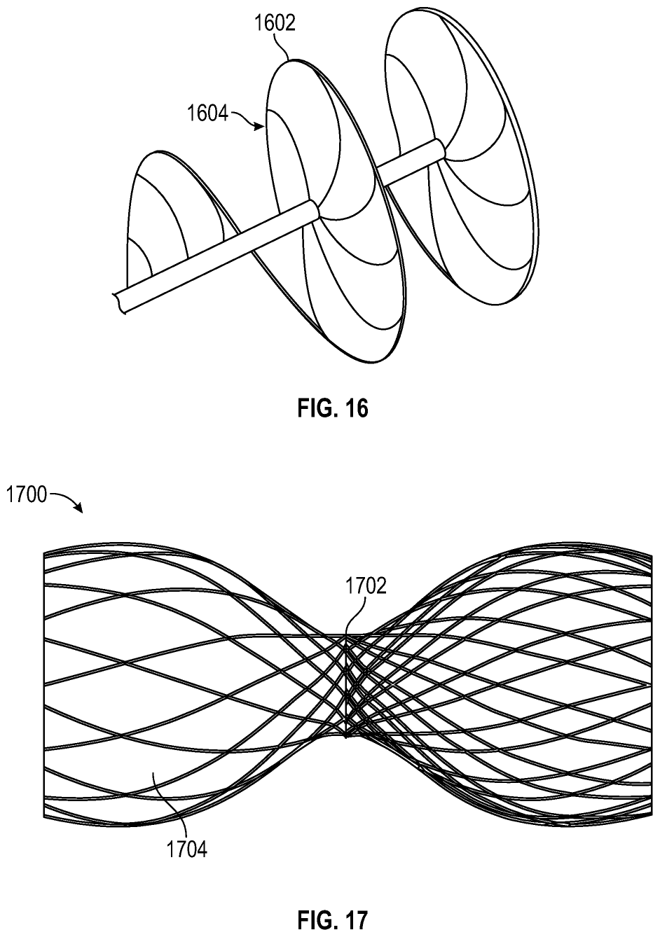

In some embodiments, the device comprises a generally funnel shaped frame, such as that described with respect to FIGS. 1A-3C. The device 1700 of FIG. 17 resembles the device like that shown in FIGS. 1A-3C, but with a second funnel-shaped stent component positioned distally to the first device such that the smaller opening ends of the devices connect, forming an hourglass shape. The second device or additional funnel shaped component can help with stabilization, providing additional anchoring.

The waist 1702 or flow restriction formed at or around the midportion can comprise dimensions similar to those described with respect to the reduced cross-sectional area opening of the devices shown in FIGS. 1A-3C.

The waist 1702 can be expanded, broken or be surgically removable to adjust the restriction.

In some embodiments, the device 1700 comprises a covering 1704 (e.g., ePTFE covering) on all or a portion of the outer surface of the device. A covering at the outer portions of the device that come into contact with the vessel wall aids in removal of the device. The device can also have a covering on all or a portion of the inner surface of the device. In some embodiments, only one half (e.g., the proximal half) of the device comprises a covering.

It will be appreciated that features described with respect to particular embodiments are also contemplated for inclusion or use with other embodiments described herein.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately.” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for restricting flow in a blood vessel, the device comprising
a first end;
a second end;
a first opening positioned at the first end, a size of the first opening adjustable from a first size to a second size;
a second opening positioned at the second end, the second opening having a greater cross-sectional area than the first opening; and
a plurality of struts extending from the first end toward the second end and defining the first opening and the second opening, the plurality of struts extending in a radially outward direction along a portion of their length,
wherein the device is collapsible to enable percutaneous delivery.

2. The device of claim 1, wherein the device is collapsible to enable percutaneous delivery through a 5 F or smaller sheath.

3. The device of any of claim 1 or 2, wherein placement of the device results in a greater than 30% reduction in pulmonary blood flow.

4. The device of any of claims 1-3, wherein the device comprises a length less than about 10 mm.

5. The device of any of claims 1-4, wherein the device is configured for placement in branch pulmonary arteries.

6. The device of any of claims 1-5, wherein the plurality of struts extend radially outwardly or longitudinally from the first end to the second end.

7. The device of any of claims 1-6, wherein the plurality of struts define an opening having a larger diameter than the blood vessel to anchor the device in place.

8. The device of any of claims 1-7, further comprising a membrane covering a portion of an outer surface of the device.

9. The device of any of claims 1-8, further comprising a membrane covering an outer surface of the device.

10. The device of any of claims 1-9, further comprising a membrane covering an inner surface of the device.

11. The device of any of claims 1-10, further comprising a membrane covering an outer surface of the device, wherein the membrane covers a portion of the device configured to contact tissue within the blood vessel.

12. The device of claim 8-11, wherein the membrane comprises ePTFE.

13. The device of any of claims 1-12, wherein placement of the device results in Qp:Qs ratios of between 0.8:1 and 2:1.

14. The device of any of claims 1-13, wherein placement of the device results in arterial oxygen saturations between about 70%-90%.

15. The device of any of claims 1-14, wherein the first end is the distal end.

16. The device of any of claims 1-14, wherein the first end is the proximal end.

17. The device of any of claims 1-16, wherein the first opening is expandable.

18. The device of any of claims 1-17, wherein the first opening is balloon expandable.

19. The device of any of claims 1-18, wherein the first opening is configured to expand upon absorption of a bioabsorbable component.

20. The device of any of claims 1-19, wherein the first opening is tethered to a portion of the frame that expands upon growth of the blood vessel, and wherein expansion of the portion of the frame causes expansion of the tethered first opening.

21. The device of any of claims 1-20, wherein the device is generally funnel shaped.

22. The device of any of claims 1-21, wherein the first opening is adjustable from an approximate diameter of about 1-3 mm to about 2-5 mm.

23. The device of any of claims 1-22, wherein the first opening is adjustable from an approximate diameter of about 1-3 mm to a diameter of the second opening.

24. The device of any of claims 1-23, wherein a diameter of the second opening is about 5-12 mm.

25. The device of any of claims 1-24, wherein a profile of the device extends slightly radially outwardly or longitudinally from the second end for a first segment and extends radially inward along a second segment from the second segment.

26. The device of claim 25, wherein the profile of the device extends generally longitudinally towards the first end from the second segment.

27. The device of any of claims 1-26, further comprising a flange positioned around the second opening.

28. The device of any of claims 1-27, further comprising a plastically deformable component positioned at or around the first opening.

29. The device of claim 28, wherein the plastically deformable component comprises a polymer and/or metal alloy.

30. The device of claim 28, wherein the plastically deformable component comprises stainless steel.

31. The device of any of claims 1-30, wherein the device is percutaneously removable.

32. The device of any of claims 1-31, wherein the device is configured to be positioned at a bifurcation between two branch blood vessels.

33. The device of any of claims 1-32, wherein the plurality of struts are woven or braided.

34. The device of any of claims 1-32, wherein the plurality of struts are laser cut.

35. The device of any of claims 1-34, wherein the first opening is configured to self-expand as the blood vessel grows larger.

36. A method for reducing flow in a blood vessel, the method comprising
percutaneously advancing a device comprising a plurality of struts disposed between a first end and a second end of the device through the vasculature to a placement site within the blood vessel; and
expanding the device from a collapsed configuration to a radially expanded configuration, the device in the radially expanded comprising a first opening at the first end and a second opening at the second end, the second opening comprising a different cross-sectional area than the first opening,
wherein the cross-sectional area of the second opening is adjustable.

37. The method of claim 36, further comprising percutaneously adjusting the cross-sectional area of the second opening.

38. The method of claim 37, wherein adjusting the cross-sectional area comprises increasing the cross-sectional area.

39. The method of claim 37, wherein adjusting the cross-sectional area comprises reducing the cross-sectional area.

40. The method of any of claims 36-39, wherein percutaneously advancing the device comprises advancing the device through a sheath with a diameter of 5F or less.

41. The method of any of claims 36-40, further comprising percutaneously removing the device.

42. The method of any of claims 36-41, wherein the placement site is a pulmonary artery.

43. The method of any of claims 36-42, wherein the placement site is a branch pulmonary artery.

44. The method of any of claims 36-43, wherein placement of the device results in Qp:Qs ratios of between 0.8:1 and 2:1.

45. The method of any of claims 36-44, wherein placement of the device results in arterial oxygen saturations between about 70%-90%.

46. The method of any of claims 36-45, wherein adjusting the cross-sectional area comprises straightening struts of the device positioned at or around the second opening.

47. The method of any of claims 36-46, wherein adjusting the cross-sectional area of the second opening comprises using a balloon to expand the second opening.

48. The method of any of claims 36-47, wherein adjusting the cross-sectional area of the second opening comprises applying energy to the second opening to expand the second opening.

49. The method of any of claims 36-48, wherein expanding the device results in positioning the device at a bifurcation of the blood vessel.

50. The method of any of claims 36-49, further comprising the device self-adjusting as the blood vessel grows and the device expands, increasing the size of the opening.

51. A device for restricting flow in a blood vessel, the device comprising
a first end;
a second end;
a first opening positioned at the first end, a size of the first opening adjustable from a first size to a second size;
a second opening positioned at the second end, the second opening having a greater cross-sectional area than the first opening; and
a plurality of struts extending from the first end toward the second end, the plurality of struts extending in a radially outward direction along a portion of their length,
wherein the device is collapsible to enable percutaneous delivery,
and wherein the device is configured to be positioned at a bifurcation between two branch pulmonary arteries.

52. The device of claim 51, wherein the first end of the device is configured to be positioned against a junction between the two branch pulmonary arteries.

53. The device of claim 51 or 52, wherein the second end of the device is configured to be positioned within a main branch upstream of the bifurcation.

54. The device of claim 51 or 52, wherein the second end of the device is configured to be positioned within a main branch downstream of the bifurcation.

55. The device of claim 51 or 52, wherein the second end of the device is configured to be positioned within a main branch at the bifurcation.

56. The device of any of claims 51-56, wherein the device is collapsible to enable percutaneous delivery through a 5 F or smaller sheath.

57. The device of any of claims 51-56, wherein placement of the device results in a greater than 30% reduction in pulmonary blood flow.

58. The device of any of claims 51-57, wherein the device comprises a length less than about 10 mm.

59. The device of any of claims 51-58, wherein the device is configured for placement in branch pulmonary arteries.

60. The device of any of claims 51-59, wherein the plurality of struts extend radially outwardly or longitudinally from the first end to the second end.

61. The device of any of claims 51-60, further comprising a membrane covering a portion of an outer surface of the device.

62. The device of any of claims 51-61, further comprising a membrane covering an outer surface of the device.

63. The device of any of claim 51-62, further comprising a membrane covering an outer surface of the device, wherein the membrane covers a portion of the device configured to contact tissue within the blood vessel.

64. The device of any of claims 51-63, wherein the first opening is expandable.

65. The device of any of claims 51-64, wherein the first opening is balloon expandable.

66. The device of any of claims 51-65, wherein the device is percutaneously removable.

67. The device of any of claims 51-66, wherein the first opening is configured to self-expand as the blood vessel grows larger.

68. The device of any of claims 51-67, wherein the plurality of struts are woven.

69. The device of any of claims 51-67, wherein the plurality of struts are laser cut.

70. A device for restricting flow in a blood vessel, the device comprising
a proximal end;
a distal end;
a plurality of struts extending from the proximal end toward the distal end, the plurality of struts configured to anchor against walls of the blood vessel; and
a membrane structure extending from or near the proximal end of the device, the membrane structure comprising a membrane structure proximal end and a membrane structure distal end, wherein the membrane structure provides a passage for blood flow within the device, and wherein the passage has a greater cross-sectional area at the membrane structure proximal end than at the membrane structure distal end, and wherein the cross-sectional area of the membrane structure distal end is adjustable.

71. The device of claim 70, wherein the device is collapsible to enable percutaneous delivery through a 5 F or smaller sheath.

72. The device of claim 70 or 71, wherein placement of the device results in a greater than 30% reduction in pulmonary blood flow.

73. The device of any of claims 70-72, wherein the device comprises a length less than about 10 mm.

74. The device of any of claims 70-73, wherein the device is configured for placement in branch pulmonary arteries.

75. The device of any of claims 70-74, wherein the plurality of struts extend radially outwardly or longitudinally from the first end to the second end.

76. The device of any of claims 70-75, further comprising a membrane covering a portion of an outer surface of the device.

77. The device of any of claims 70-76, further comprising a membrane covering an outer surface of the device.

78. The device of any of claims 70-77, further comprising a membrane covering an outer surface of the device, wherein the membrane covers a portion of the device configured to contact tissue within the blood vessel.

79. The device of any of claims 70-78, wherein the distal end of the device is configured to be positioned against a junction between the two branch pulmonary arteries.

80. The device of any of claims 70-79, wherein the proximal end of the device is configured to be anchored within a blood vessel upstream of the bifurcation.

81. The device of any of claims 70-80, the membrane structure further comprising a support structure positioned at or near a membrane structure distal end.

82. The device of claim 81, wherein the support structure is plastically deformable.

83. The device of claim 81, wherein the support structure forms a flow restricting portion of the passage into an ovular or duckbill shape.

84. The device of any of claims 70-83, the membrane structure further comprising a support ring positioned at or near a membrane structure distal end.

85. The device of claim 84, wherein the support ring is expandable.

86. The device of any of claims 70-85, wherein the membrane structure comprises a valve positioned within the passage.

87. The device of claim 86, wherein the device comprises a slit, cross-slit, or duckbill valve.

88. The device of any of claims 70-87, wherein the membrane structure generally comprises a funnel shape.

89. The device of any of claims 70-88, wherein the membrane structure is sufficiently flexible to change orientation due to fluid flow.

90. The device of any of claims 70-89, wherein the membrane structure distal end comprises a diameter of about 1-3 mm.

91. The device of any of claims 70-90, wherein the device is percutaneously removable.

92. The device of any of claims 70-91, wherein the membrane structure folds over an end of the plurality of struts and extends towards another end of the plurality of struts, forming a covering.

93. The device of any of claims 70-92, wherein the first opening is configured to self-expand as the blood vessel grows larger.

94. A method for reducing flow in a blood vessel, the method comprising
percutaneously advancing a device through the vasculature to a placement site within the blood vessel;
expanding an anchor portion of the device such that it bears against an inner wall of the blood vessel at the placement site;
expanding a membrane structure of the device, the membrane structure connected to the anchor portion of the device at or near a proximal portion of the anchor portion, the membrane structure forming a passageway for blood flow through the device, expanding comprising:
expanding a proximal end of the membrane structure to form a proximal opening of the passageway; and
expanding a distal end of the membrane structure to form a distal opening of the passageway, the distal opening comprising a smaller cross-sectional area than the proximal opening of the passageway,
wherein the cross-sectional area of the distal opening is adjustable.

95. The method of claim 94, further comprising percutaneously adjusting the cross-sectional area of the distal opening.

96. The method of claim 95, wherein adjusting the cross-sectional area comprises increasing the cross-sectional area.

97. The method of claim 95, wherein adjusting the cross-sectional area comprises reducing the cross-sectional area.

98. The method of any of claims 94-97, wherein percutaneously advancing the device comprises advancing the device through a sheath with a diameter of 5F or less.

99. The method of any of claims 94-98, further comprising removing the device.

100. The method of any of claims 94-99, wherein the placement site is a pulmonary artery.

101. The method of any of claims 94-100, wherein the placement site is a branch pulmonary artery.

102. The method of any of claims 94-101, wherein placement of the device results in Qp:Qs ratios of between 0.8:1 and 2:1.

103. The method of any of claims 94-102, wherein placement of the device results in arterial oxygen saturations between about 70%-90%.

104. The method of any of claims 94-103, wherein adjusting the cross-sectional area comprises expanding a strut of the device positioned at or around the second opening.

105. The method of any of claims 94-104, wherein adjusting the cross-sectional area of the second opening comprises using a balloon to expand the second opening.

106. The method of any of claims 94-105, wherein adjusting the cross-sectional area of the second opening comprises applying energy to expand the second opening.

107. The method of any of claims 94-106, further comprising the second opening self expanding as the blood vessel grows.

108. A device for reducing blood flow in a blood vessel of a pediatric patient, comprising
an anchor portion configured to anchor against a wall of the blood vessel; and
an obstruction connected to the anchor portion and positioned such that it blocks blood flow within the blood vessel,
wherein the anchor portion is configured to expand as the blood vessel grows, increasing the flow path around the obstruction.

109. The device of claim 108, wherein the device is collapsible to enable percutaneous delivery through a 5 F or smaller sheath.

110. The device of claim 108 or 109, wherein placement of the device results in a greater than 30% reduction in pulmonary blood flow.

111. The device of any of claims 108-110, wherein the device comprises a length less than about 10 mm.

112. The device of any of claims 108-111, wherein the device is configured for placement in branch pulmonary arteries.

113. The device of any of claims 108-112, further comprising a membrane covering a portion of an outer surface of the device.

114. The device of any of claims 108-113, further comprising a membrane covering an outer surface of the device.

115. The device of any of claims 108-114, further comprising a membrane covering an inner surface of the device.

116. The device of any of claims 108-115, further comprising a membrane covering an outer surface of the device, wherein the membrane covers a portion of the device configured to contact tissue within the blood vessel.

117. The device of any of claims 108-116, wherein the membrane comprises ePTFE.

118. The device of any of claims 108-117, wherein placement of the device results in Qp:Qs ratios of between 0.8:1 and 2:1.

119. The device of any of claims 108-118, wherein placement of the device results in arterial oxygen saturations between about 70%-90%.

120. The device of any of claims 108-119, wherein the anchor portion comprises a plurality of woven struts.

121. The device of any of claims 108-119, wherein the anchor portion comprises a plurality of laser cut struts.

122. The device of any of claims 108-121, wherein a cross-sectional area of the flow path can be expanded using application of energy.

123. A method for reducing flow in a blood vessel, the method comprising
percutaneously advancing a device comprising an anchor portion supporting an obstruction through the vasculature to a placement site within the blood vessel; and
expanding the anchor portion from a collapsed configuration to a radially expanded configuration to anchor to the blood vessel at the placement site and to define a flow path for blood around the obstruction,
wherein the anchor portion is configured to self-expand as the blood vessel grows, increasing a cross-sectional area of the flow path around the obstruction.

124. The method of claim 123, further comprising percutaneously removing the device.

125. The method of any of claim 123 or 124, wherein the placement site is a pulmonary artery.

126. The method of any of claims 123-125, wherein the placement site is a branch pulmonary artery.

127. The method of any of claims 123-126, wherein placement of the device results in Qp:Qs ratios of between 0.8:1 and 2:1.

128. The method of any of claims 123-127, wherein placement of the device results in arterial oxygen saturations between about 70%-90%.
